(19) United States
(12) Patent Application Publication
(10) Pub. No.: US 2022/0240965 A1
(43) Pub. Date: Aug. 4, 2022

(54) ACNE EXTRACTION TOOL DEVICE

(71) Applicant: Naomi Marshall, Saginaw, MI (US)

(72) Inventor: Naomi Marshall, Saginaw, MI (US)

(21) Appl. No.: 17/555,721

(22) Filed: Dec. 20, 2021

Related U.S. Application Data
(60) Provisional application No. 63/145,725, filed on Feb. 4, 2021.

(51) Int. Cl. A61B 17/30

(52) U.S. Cl. CPC A61B 17/30; A61B 2017/00747; A61B 2017/308

(57) ABSTRACT

The present invention relates to an acne extraction tool device comprised of a body that has a first member, a second member, at least one hinge, at least one spring and a cup member. The cup member can be placed on the skin of a user and over the acne to be extracted. A user can then grip each member and press the cup downwards onto the skin, while simultaneously pushing each member towards one another, such that the cup becomes contorted. As a result, the cup applies uniform pressure around the acne to extract the acne. The extracted bodily matter from the acne is then collected on the interior surface of the cup, such that it cannot contact any nearby surfaces.

### CROSS-REFERENCE TO RELATED APPLICATION

The present application claims priority to, and the benefit of, U.S. Provisional Application No. 63/145,725, which was filed on Feb. 4, 2021 and is incorporated herein by reference in its entirety.

### FIELD OF THE INVENTION

The present invention relates generally to the field of acne extraction tools. More specifically, the present invention relates to an acne extraction tool device comprised of a body that has a first member, a second member, at least one hinge, at least one spring and a cup member. The first member and the second member are preferably identical in structure and are preferably generally cylindrical in shape. The cup member can be placed on the skin of a user and over the acne to be extracted. A user can then grip each member in their hand with their thumb and index finger and press the cup downwards onto their skin, while simultaneously pushing each member towards one another such that the cup becomes contorted. As a result, the cup applies uniform pressure around the acne to extract the acne. Accordingly, the present disclosure makes specific reference thereto. Nonetheless, it is to be appreciated that aspects of the present invention are also equally applicable to other like applications, devices and methods of manufacture.

### BACKGROUND

Many individuals suffer from differing types of acne in the form of blackheads, whiteheads, papules, pustules, nodules, cysts and other forms of blemishes on various skin surfaces on their body. Because said blemishes/acne are unsightly, a user may wish to remove them from their body. In addition, if left in the skin for a long period of time acne can cause significant skin problems, that must then be surgically removed. Further, improperly removing acne can result in permanent scarring of the skin. In addition, when acne is squeezed, bodily matter may shoot out of the acne and onto nearby surfaces, which is undesirable and unsanitary. Many users also attempt to remove acne with their fingers, which leads to improper extraction which can damage the skin and also create permanent scarring.

Therefore, there exists a long-felt need in the art for a tool that allows a user to properly extract acne. There also exists a long-felt need in the art for an acne extraction tool device that allows a user to quickly and easily extract acne. Further, there exists a long-felt need in the art for an acne extraction tool device that prevents bodily matter from contacting nearby surfaces, when acne is extracted. Finally, there exists a long-felt need in the art for an acne extraction tool device that allows a user to properly and safely extract acne, in a manner that does not create scarring.

The subject matter disclosed and claimed herein, in one embodiment thereof, comprises an acne extraction tool device. The device is comprised of a body that has a first member, a second member, at least one hinge, at least one spring and a cup member. The first member and the second member are preferably identical in structure and are preferably generally cylindrical in shape. The cup member can be placed on the skin of a user and over the acne/blemish that is to be extracted. The cup member is comprised of a flat bottom surface with a continuous opening that can be placed around the acne, such that the cup encloses the acne. A user can then grip each member in their hand with their thumb and index finger and press the cup downwards onto their skin and simultaneously push each member towards one another such that the cup becomes contorted. As a result, the cup applies uniform pressure on the acne, to extract the acne is a safe manner. The extracted bodily matter from the acne is then collected on the interior surface of the cup, such that it cannot contact any nearby surfaces.

In this manner, the acne extraction tool device of the present invention accomplishes all of the forgoing objectives and provides an improved acne extraction tool device. The device allows a user to properly extract acne in a quick and safe manner. Further, the cup of the device prevents bodily matter from contacting nearby surfaces when the acne is extracted. Finally, the device allows a user to properly and safely extract acne, in a manner that does not create scarring.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises an acne extraction tool device primarily comprised of a body that has a first member, a second member, at least one hinge, at least one spring and a cup member. The first member and the second member are preferably identical in structure and are preferably generally cylindrical in shape. Each member may further have a textured grip, to allow a user to more easily grip each member. Both members meet at their second ends and are preferably connected via at least one hinge. In addition, a spring may be fixedly or removably attached to the second end of each member, between the members. The spring allows the members to move closer to one another, when the members are squeezed by the thumb and index finger of a user. In one embodiment, it is further contemplated that the spring may be within the hinge itself, wherein the hinge is spring loaded.

The device is further comprised of a cup member that can be placed on the skin of a user and over the acne/blemish that is about to be extracted. The body of the cup is preferably flexible, or semi-flexible and may be disposable or reusable in differing embodiments. The body is further comprised of a flat top surface and a flat bottom surface with a continuous opening that can be placed around acne, such that the cup encloses the acne. The side surfaces of the cup are preferably angled such that the cup forms a generally trapezoidal shape, and wherein the side surfaces are comprised of at least one channel that removably receives the first end of each member to temporarily secure the members within the body, while the device is being used to extract acne.

The invention is also comprised of a method to allow a user to safely extract acne using the device. To do so, a user first places each member into each channel, on the side surfaces of the cup member. Then, the cup member is placed on the skin of a user, such that the continuous opening is placed around the acne that a user wishes to extract. Next, a user grips each member in his or her hand with the thumb and index finger, and presses the cup downwards onto the skin, while simultaneously pushing each member towards one another. As a result, the cup body becomes contorted (i.e. the members push inwards on the cup body), such that it applies uniform pressure on the acne to safely extract the acne. The extracted bodily matter from the acne is then collected on the interior surface of the cup body, such that it cannot contact any nearby surfaces. Finally, a user can remove the cup from the members and discard or clean the cup as necessary.

Accordingly, the acne extraction tool device of the present invention is particularly advantageous as it allows a user to properly extract acne in a quick and safe manner. Further, the cup of the device prevents bodily matter from contacting nearby surfaces, when acne is extracted. Finally, the device allows a user to properly and safely extract acne, in a manner that does not create scarring.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a top perspective view of one potential embodiment of the acne extraction tool device of the present invention in accordance with the disclosed architecture;

FIG. 2 illustrates a front perspective view of one potential embodiment of the acne extraction tool device of the present invention in use in accordance with the disclosed architecture; and

FIG. 3 illustrates a flow chart of one potential embodiment of a method of using the acne extraction tool device of the present invention in accordance with the disclosed architecture to alleviate acne.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a tool that allows a user to properly extract acne. There also exists a long-felt need in the art for an acne extraction tool device that allows a user to quickly and easily extract acne. Further, there exists a long-felt need in the art for an acne extraction tool device that prevents bodily matter from contacting nearby surfaces when acne is extracted, and that allows a user to properly and safely extract acne in a manner that does not create scarring.

The present invention, in one exemplary embodiment, is comprised of an acne extraction tool device. The device primarily comprises a body that has a first member, a second member, at least one hinge, at least one spring and a cup member. The first member and the second member are preferably identical in structure and are preferably generally cylindrical in shape. Each member may further have a textured grip to allow a user to more easily grip each member. The first and the second members meet at their second ends and are preferably connected via at least one hinge. A spring may also be fixedly or removably-attached to the second end of each member between the members, wherein the spring allows the members to move closer to one another when the members are squeezed by the thumb and index finger of a user. In a further embodiment, the spring may be within the hinge itself, wherein the hinge is spring-loaded.

The device also comprises a cup member that can be placed on the skin of a user and over the acne/blemish that the user wishes to extract. The body of the cup is preferably flexible, or semi-flexible and may be disposable or reusable in differing embodiments. In addition, the body is comprised of a flat top surface and a flat bottom surface with a continuous opening that can be placed around the acne, such that the cup encloses the acne. The side surfaces of the cup are preferably angled, such that the cup forms a generally trapezoidal shape. Further, the side surfaces are comprised of at least one channel that removably receives the first end of each member to temporarily secure the members within the body while the device is being used to extract acne.

The invention also discloses a method of use to allow a user to safely extract acne using the device. A user first places each member into each channel on the side surfaces of the cup body. Then, the cup member is placed on the skin of a user, such that the continuous opening is placed around the acne a user wishes to extract. Next, a user grips each member in his or her hand with the thumb and index finger, presses the cup downwards onto the skin and simultaneously pushes each member towards one another. As a result, the cup becomes contorted (i.e. the members push inwards on the body), such that it applies uniform pressure on the acne to extract the acne. The extracted bodily matter from the acne is then collected on the interior surface of the body, such that it cannot contact any nearby surfaces of the user. Finally, a user can remove the cup from the members and discard or clean the cup as necessary.

Accordingly, the acne extraction tool device of the present invention is particularly advantageous as it allows a user to properly extract acne in a quick and safe manner. Further, the cup of the device prevents bodily matter from contacting nearby surfaces when the acne is extracted. Finally, the device allows a user to properly and safely extract acne, in a manner that does not create scarring.

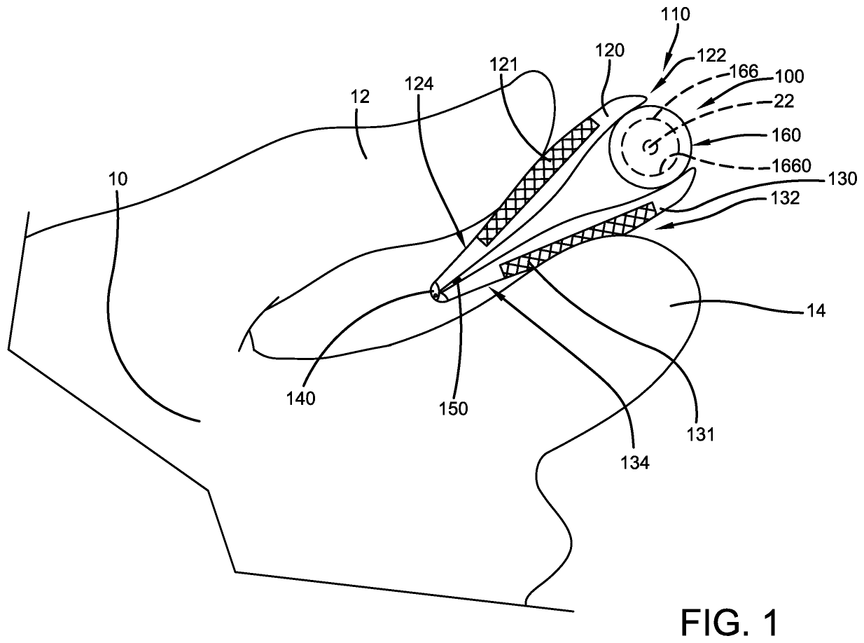

Referring initially to the drawings, FIG. 1 illustrates a top perspective view of one embodiment of the acne extraction tool device 100 of the present invention. The device 100 is primarily comprised of a body 110 that has a first member 120, a second member 130, at least one hinge 140, at least one spring 150 and a cup member 160. In differing embodiments, all components of the device 100, unless otherwise specified, may be comprised of a durable metal, such as but not limited to, stainless steel or aluminum, etc., or any other suitable metal as is known in the art. In addition, all components of the device 100, unless otherwise specified, may be comprised of a plastic material, such as but not limited to, acrylic, polycarbonate, polyethylene, thermoplastic, acrylonitrile butadiene styrene, low density polyethylene, medium density polyethylene, high density polyethylene, polyethylene terephthalate, polyvinyl chloride, polystyrene, polylactic acid, acetal, nylon, fiberglass, etc., or any other suitable plastic material as is known in the art that may be flexible, semi-flexible or rigid in differing embodiments.

The first member 120 and the second member 130 are preferably identical in structure and are preferably generally cylindrical in shape. However, in differing embodiments the members 120, 130 may be of any shape known in the art, such that they can easily be grasped by the thumb 12 and index finger 14 of a user. To further facilitate easy grasping by a user, each member 120, 130 may have a textured grip 121, 131 that in differing embodiments may be knurled or comprised of an additional rubber grip surface that may be removable or fixedly-attached. Thus, a user grips each member 120, 130 in his or her hand 10 with the thumb 12 and index finger 14 (or other fingers).

Furthermore, both members 120, 130 meet at their second ends 124, 134, wherein the members 120, 130 are preferably connected via at least one hinge 140. In differing embodiments, the hinge 140 may be any hinge type known in the art, such as but not limited to: an offset blind hinge, a knuckle hinge, a butt hinge, a rising butt hinge, a gravity pivot hinge, a ball bearing hinge, a barrel hinge, a concealed hinge, a knife hinge, a piano hinge, a strap hinge, a pivot hinge, a gas-piston hinge, an injection molded hinge, a locking hinge, etc. In addition, a spring 150 of any suitable spring-type known in the art may be fixedly or removably-attached to the second end 124, 134 of each member 120, 130 between the members 120, 130. The spring 150 allows the members 120, 130 to move closer to one another when the members 120, 130 are squeezed by the thumb 12 and index finger 14 of a user's hand 10, such that a compression force is created within the spring 150. In one embodiment, it is further contemplated that the spring 150 may be within the hinge 140 itself, wherein the hinge 140 is spring-loaded.

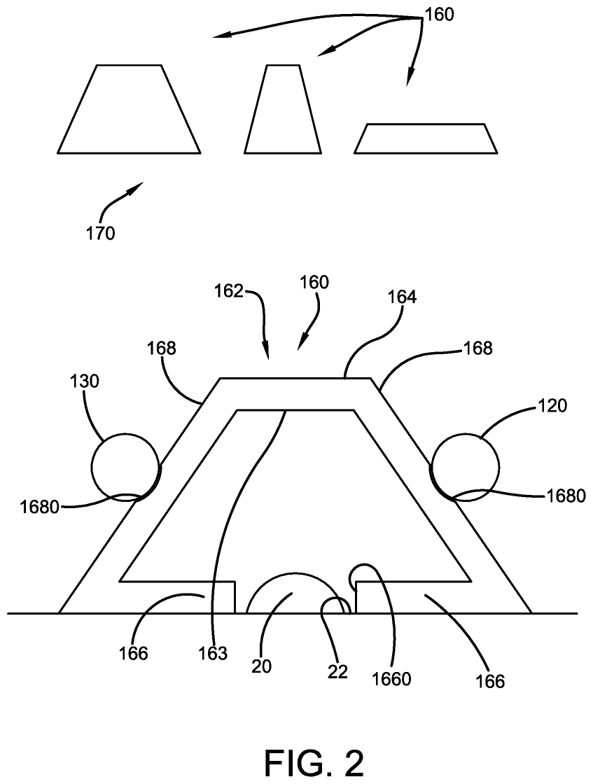

FIG. 2 illustrates a front perspective view of one embodiment of the acne extraction tool device 100 of the present invention in use. The device 100 comprises a cup member 160 that can be placed on the skin 20 of a user and over the acne/blemish 22 that is about to be extracted. The body 162 of the cup 160 is preferably flexible or semi-flexible. In differing embodiments, the device 100 may also be comprised of a kit 170, which would contain a plurality of differing cup members 160 of various sizes to allow the cup 160 to be placed over and adequately cover acne 22 of various sizes and in various body locations. In addition, the cup 160 may be disposable or reusable. The body 162 is further comprised of a flat top surface 164 and a flat bottom surface 166 with a continuous opening 1660 that can be placed around the acne 22, such that the cup 160 encloses the acne 22. The side surfaces 168 of the cup 160 are preferably angled, such that the cup 160 forms a generally trapezoidal shape. However, in differing embodiments the cup 160 may be circular, rectangular, square, triangular, octagonal, hexagonal, etc., in shape or any other suitable shape as is known in the art. The side surfaces 168 of the body 162 are further comprised of at least one channel 1680, that removably receives the first end 122, 132 of each member 120, 130. In this regard, the members 120, 130 are temporarily secured within the body 162, while the device 100 is being used to extract acne 22. However, in differing embodiments the first end 122, 132 of each member may be fixedly-attached within the channels 1680 and not removable.

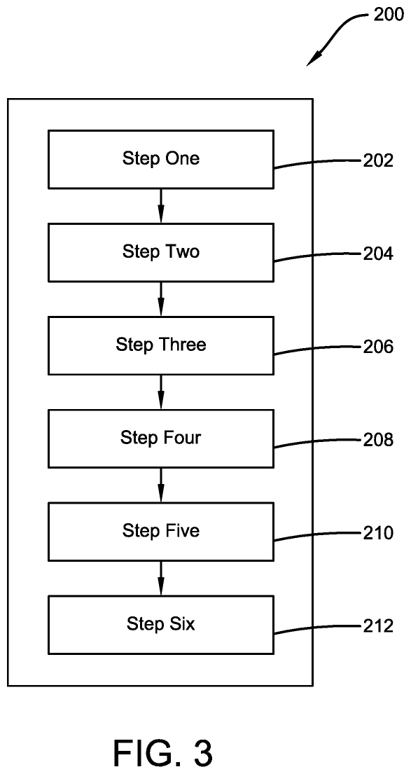

FIG. 3 illustrates a flow chart of one embodiment of the method 200 of using the acne extraction tool device 100 of the present invention. As previously noted, the device 100 can be used to allow a user to safely extract acne 22. To do so, at 202 a user first places each member 120, 130 into each channel 1680 on the side surfaces 168 of the device 100. Then at 204, the cup member 160 is placed on the skin 20 of a user, wherein the continuous opening 1660 is placed around the acne 22 a user wishes to extract. Next at 206, a user grips each member 120, 130 in his or her hand 10 with the thumb 12 and index finger 14 (or other fingers). At 208, a user then presses the cup 160 downwards onto the skin 20, and simultaneously pushes each member 120, 130 towards one another. In this manner, the body 162 of the cup 160 becomes contorted (i.e. the members 120, 130 push inwards on the body 162), due to the pressure of each member 120, 130 and the compression of the spring 150 on the cup 160, wherein the continuous opening 1160 of the cup 160 applies uniform pressure around the acne 22. At 210, the extracted bodily matter from the acne 22 is then collected on the interior surface 163 of the body 162. As a result, the bodily matter cannot contact any nearby surfaces. Then at 212, a user can remove the cup 160 from the members 120, 130 and discard or clean the cup 160 as necessary. In one embodiment, the acne extraction tool device 100 may further comprise an anti-microbial coating to prevent or reduce the likelihood of infection from an extraction procedure.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “acne extraction tool device” and “device” are interchangeable and refer to the acne extraction tool device 100 of the present invention.

Notwithstanding the forgoing, the acne extraction tool device 100 of the present invention and its various components can be of any suitable size and configuration as is known in the art without affecting the overall concept of the invention, provided that they accomplish the above-stated objectives. One of ordinary skill in the art will appreciate that the size, configuration and material of the acne extraction tool device 100 as shown in FIGS. 1-3 is for illustrative purposes only, and that many other sizes and shapes of the acne extraction tool device 100 are well within the scope of the present disclosure. Although the dimensions of the acne extraction tool device 100 are important design parameters for user convenience, the acne extraction tool device 100 may be of any size, shape and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An acne extraction tool device for extracting acne, the acne extraction tool device comprising:
a body comprised of a first member and a second member, wherein the first member and the second member each comprise a first end and a second end, and further wherein the second end of both the first member and the second member are connected together via at least one hinge and at least one spring; and
a cup member comprising a top surface, a bottom surface with a continuous opening, a side surface, and an interior surface, wherein the cup member is placed on a user's skin, such that the continuous opening is placed over acne a user wants to extract, and further
wherein the first member and the second member are gripped by a user's thumb and index finger and then pressed on the cup member, creating uniform pressure around the acne to extract it.

2. The acne extraction tool device of claim 1, wherein the first member and the second member can be squeezed toward each other via the at least one hinge and the at least one spring to contort the cup member, such that bodily matter is extracted from the acne and collected on the interior surface of the cup member.

3. The acne extraction tool device of claim 2, wherein the cup member is disposable after use.

4. The acne extraction tool device of claim 2, wherein the cup member is reusable and can be cleaned after use.

5. An acne extraction tool device for extracting acne comprising:
a body comprised of a first member and a second member;
wherein the first member and the second member each comprise a first end and a second end;
wherein the second end of both the first member and the second member are connected together via at least one hinge and at least one spring; and
wherein the first and the second members comprise a grip component;
a cup member comprising a top surface, a bottom surface with a continuous opening, an interior surface, and a side surface with a channel that receives the first and the second members;
wherein the cup member is placed on a user's skin, such that the continuous opening is placed over acne a user wants to extract; and
wherein the first member and the second member are each positioned in the channel and gripped by a user's thumb and index finger and then pressed on the cup member, creating uniform pressure around the acne to extract it.

6. The acne extraction tool device of claim 5, wherein the grip component of the first member and the grip component of the second member is a removable rubber grip.

7. The acne extraction tool device of claim 5, wherein the grip component of the first member and the grip component of the second member is a knurled, textured grip.

8. The acne extraction tool device of claim 5, wherein the first member and the second member can be squeezed toward each other via the at least one hinge and the at least one spring to contort the cup member, such that bodily matter is extracted from the acne and collected on the interior surface of the cup member.

9. The acne extraction tool device of claim 8, wherein the at least one hinge is a pivot hinge that connects the first member and the second member together at the second ends.

10. The acne extraction tool device of claim 9, wherein the at least one spring is positioned between the first member and the second member in a same position as the at least one hinge.

11. The acne extraction tool device of claim 10, wherein the at least one spring allows the first member and the second member to move closer to one another, when both the first and the second members are squeezed by a user, such that a compression force is created within the at least one spring.

12. The acne extraction tool device of claim 5, wherein the at least one hinge is a spring-loaded hinge.

13. The acne extraction tool device of claim 5, wherein the cup member is a flexible member.

14. The acne extraction tool device of claim 5, wherein the cup member is a semi-rigid member.

15. The acne extraction tool device of claim 5, wherein the cup member is disposable after use.

16. The acne extraction tool device of claim 5, wherein the cup member is reusable and can be cleaned after use.

17. The acne extraction tool device of claim 5, wherein the device is comprised of a kit comprising a plurality of cup members of differing sizes.

18. A method of using an acne extraction tool device to alleviate an acne, the method comprising the steps of:
placing a first member and a second member of the device into a channel on a side surface of a cup member of the device;
placing the cup member on a skin of a user, wherein a continuous opening of the cup member is placed around the acne;
gripping the first member and the second member using a thumb and an index finger;
pressing the cup member downwards onto the skin and simultaneously pushing the first member and the second member towards one another to contort the cup member, such that it applies uniform pressure around the acne to extract the acne;
extracting bodily matter from the acne;
collecting the bodily matter on an interior surface of the cup member; and
removing the cup member from the first and the second members.

19. The acne extraction tool device of claim 18 further comprising a step of discarding the cup member once the extraction is complete.

20. The acne extraction tool device of claim 18 further comprising a step of cleaning the cup member once the extraction is complete and reusing the cup member.
